(19) United States
(12) Patent Application Publication
(10) Pub. No.: US 2006/0246520 A1
(43) Pub. Date: Nov. 2, 2006

(54) Gamma delta t cell-mediated therapy

(75) Inventors: Eric Champagne, Toulouse (FR); Ronald Barbaras, Beauzelle (FR); Marc Bonneville, Vertou (FR); Emmanuel Scotet, Nantes (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: INSTITUT NATIONAL DE LA SANTE ET DE LA RECHERCHE MEDICALE, PARIS (FR)

(21) Appl. No.: 10/558,110

(22) PCT Filed: May 19, 2004
(86) PCT No.: PCT/EP04/05408

§ 371 Date: Nov. 22, 2005

Related U.S. Application Data
(60) Provisional application No. 60/472,735, filed on May 23, 2003.

(51) Int. Cl. G01N 33/574

(52) U.S. Cl. 435/7.23

(57) ABSTRACT

The present invention relates to compositions and methods for determining or modulating the activity of immune and/or target cells in vitro, ex vivo or in vivo. The invention more specifically relates to methods and compositions for determining sensitivity of cells to the activity of γδT cells, as well as to the use of these compositions and methods for patient screening or selection, therapy improvement, compound selection, etc. The invention also provides compositions and kits suitable for carrying out these methods. The methods may be used in any mammalian subject, preferably any human subject that may benefit from γδT cell-mediated therapy, including patients with tumors, immune or infectious diseases.

The present invention relates to compositions and methods for determining or modulating the activity of immune and/or target cells in vitro, ex vivo or in vivo. The invention more specifically relates to methods and compositions for determining sensitivity of cells to the activity of γδT cells, as well as to the use of these compositions and methods for patient screening or selection, therapy improvement, compound selection, etc. The invention also provides compositions and kits suitable for carrying out these methods. The methods may be used in any mammalian subject, preferably any human subject that may benefit from γδT cell-mediated therapy, including patients with tumors, immune or infectious diseases.

### INTRODUCTION

Peripheral T lymphocytes classically recognize, through their αβ T cell receptors (TCR), foreign peptidic antigens bound to class I or class II major histocompatibility complex (MHC) molecules. Besides these “conventional” T cells, other subsets expressing either αβ or γδ TCR react with a more heterogeneous set of non-peptidic compounds, either in a native form or in association with conserved MHC-related molecules (Beckman et al., 1994; Moody et al., 1997; Spada et al., 2000).

In humans, the vast majority of peripheral blood γδ T cells use a particular combination of variable regions (Vγ9 and Vδ2) to form their TCR. These Vγ9Vδ2 T cells are activated in a TCR-dependent fashion by several small phosporylated (Constant et al., 1994; Tanaka et al., 1994) or aminated (Bukowski et al., 1999) alkyl molecules. Vγ9Vδ2 T cell activation by these compounds requires intercellular contact, thus suggesting some form of antigen presentation (Lang et al., 1995; Morita et al., 1995). Vγ9Vδ2 cells also react against several fresh or cultured tumors in vitro and exhibit both cytolytic activity and production of inflammatory cytokines (TNFα, IFNγ). This activity is tightly regulated by NK-like receptors for MHC class-Ia and class Ib antigens which are prominently expressed by this T cell subset (Fisch et al., 1997; Halary et al., 1997). Thus, besides their role in immunity against viral and bacterial infections (Bukowski et al., 1994), γδ T cells are probably involved in tumor surveillance (Bukowski et al., 1995; Fisch et al., 1997; Wu et al., 2002) as also supported by in vivo experiments (Girardi et al., 2001; Malkovska et al., 1994; Malkovska et al., 1992).

Although some human γδ T cells of the Vδ1 subset react towards non classical, stress-induced MHC molecules MICA/B (Groh et al., 1998; Wu et al., 2002) through their TCR and/or activatory receptors such as NKG2D, tumor antigens recognized by Vγ9Vδ2 T cells remain unknown and strategies for activating these cells, as well as for screening target cells sensitive to γδ T cells' activity are highly needed to develop powerful therapeutic strategies.

### SUMMARY OF THE INVENTION

The present invention now discloses novel, improved strategies for generating, regulating or exploiting the activity of γδ T cells. The present invention unexpectedly shows that an entity related to the mitochondrial ATP-synthase is expressed on the membrane of target tumor cells and promotes their recognition by Vγ9Vδ2 T cells. When immobilized, purified ATP synthase induces the selective activation of this cell population. The Vγ9Vδ2 TCR and the ATP synthase also bind a delipidated form of apolipoprotein A-I as demonstrated by surface plasmon resonance. Moreover, the presence of apolipoprotein A-I in the culture medium is required for optimal activation of Vγ9Vδ2 T cells and TCR binding to tumors expressing ATP synthase. This invention thus identifies an unanticipated tumor recognition mechanism by Vγ9Vδ2 lymphocytes and allows the design of novel therapeutic and pharmacogenomic approaches.

A first aspect of this invention resides more particularly in methods of assessing the sensitivity of a pathologic cell to γδT cell activity (e.g., lysis or cytotoxicity), comprising determining in vitro or ex vivo whether said pathologic cell expresses, at the surface thereof, an ATP synthase polypeptide or a portion thereof, the cell surface expression of an ATP synthase polypeptide or a portion thereof being an indication of the sensitivity of the pathologic cell to γδT cell lysis or cytotoxicity.

An other aspect of this invention resides in methods of assessing whether a cell is a target cell for γδT cells, comprising determining in vitro or ex vivo whether said cell expresses, at the surface thereof, an ATP synthase polypeptide or a portion thereof, the cell surface expression of an ATP synthase polypeptide or a portion thereof being an indication that the cell is a target of γδT cells.

A further aspect of this invention lies in methods of assessing the responsiveness of a patient to γδT cell-mediated therapy, comprising determining whether said patient contains pathologic cells, tissues or organs that express, at the surface thereof, an ATP synthase polypeptide or a portion thereof, the cell surface expression of an ATP synthase polypeptide or a portion thereof being an indication of the responsiveness of the patient to γδT cell-mediated therapy.

The invention also relates to methods of selecting patients for a γδT cell-mediated therapy program, comprising determining whether a patient contains pathologic cells, tissues or organs that express, at the surface thereof, an ATP synthase polypeptide or a portion thereof, the cell surface expression of an ATP synthase polypeptide or a portion thereof being a criteria for selecting said patient for said γδT cell-mediated therapy program.

A further object of this invention resides in methods of stimulating γδT cells, comprising contacting γδT cells with an ATP synthase polypeptide or a TCR-binding fragment thereof. In a specific embodiment, the stimulation method uses a complex comprising an ATP synthase polypeptide or a TCR-binding fragment thereof linked to an apolipoprotein A1 polypeptide. These components (or some of them) may be immobilized on a solid support, such as a bead, plate, etc.

A further object of this invention resides in methods of stimulating γδT cells, comprising contacting γδT cells with an apolipoprotein A1 polypeptide or a variant or ATP synthase-binding fragment thereof.

The invention also provides a method of increasing sensitivity of a target cell to γδT cell lysis or cytotoxicity, comprising causing or increasing cell surface expression of an ATP synthase polypeptide or a portion thereof in said target cell. The method may be performed in vitro, ex vivo or in vivo.

The invention also relates to an improvement in methods of treating a cancer, an infectious disease, an autoimmune disease or an allergic disease in a subject by γδT cell-mediated therapy, the improvement comprising determining, prior to or during said therapy, whether said subject contains pathologic cells, tissues or organs that express, at the surface thereof, an ATP synthase polypeptide or a portion thereof, the cell surface expression of an ATP synthase polypeptide or a portion thereof being a criteria for selecting or monitoring said patient for said γδT cell-mediated therapy.

The invention also relates to methods of screening, selecting or identifying a compound, comprising:
a) separately contacting γδT cells with an ATP synthase polypeptide or a TCR-binding fragment thereof in the presence and in the absence of a candidate compound and,
b) screening, selecting or identifying a compound that modulates activation of said γδT cells by said ATP synthase polypeptide or a TCR-binding fragment thereof.

The invention is particularly suited for use with human cells or subjects, and can be used to diagnose or select cells or patients having cancers or infectious diseases sensitive to γδT cell-mediated therapy. As will be discussed further, the ATP synthase polypeptide may be an ATP synthase α subunit or β subunit or both, and the portion thereof may be an extracellular domain of an ATP synthase α or β subunit.

### LEGEND TO THE FIGURES

FIG. 1: Binding of extracellular apoA-I to tumor cell membrane.

(a) Indirect immunofluorescence staining with M5 mAb or a control IgG1 antibody (shaded histograms) of human cell lines cultured in presence of fetal calf serum (fcs). Daudi, Raji: Burkitt's lymphomas; Awells, RPMI8866: B-lymphoblastoid cell lines; RPMI 8226: B-cell myeloma; K562: erythroid leukemia; U937: monocytic leukemia; MOLT-4, Jurkat: T cell leukemias.

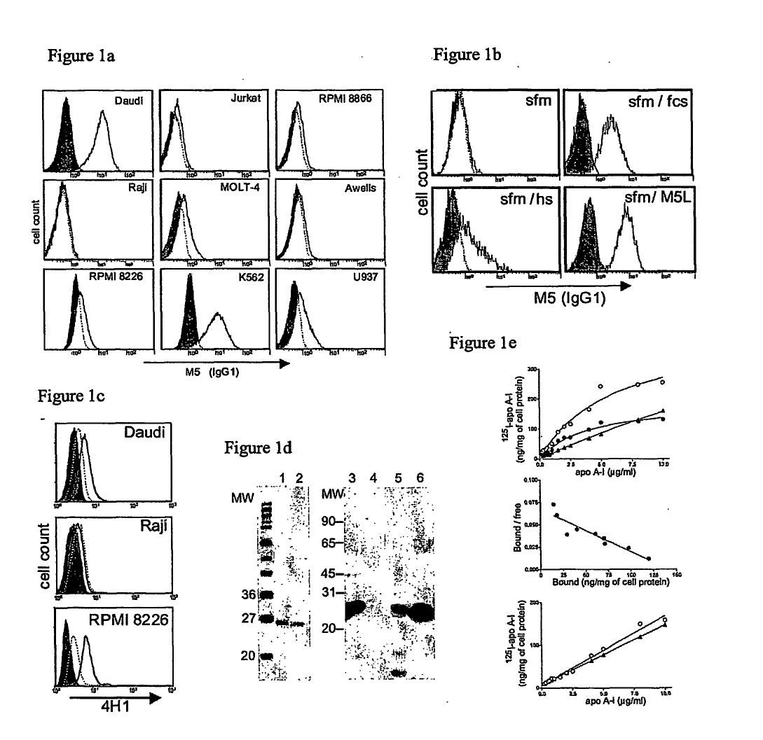

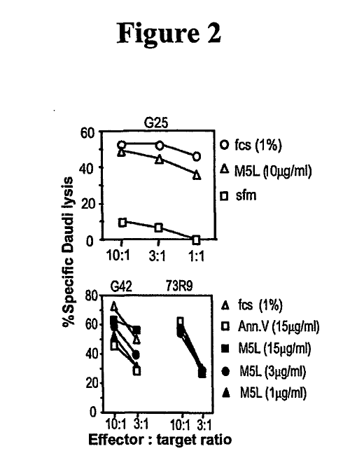

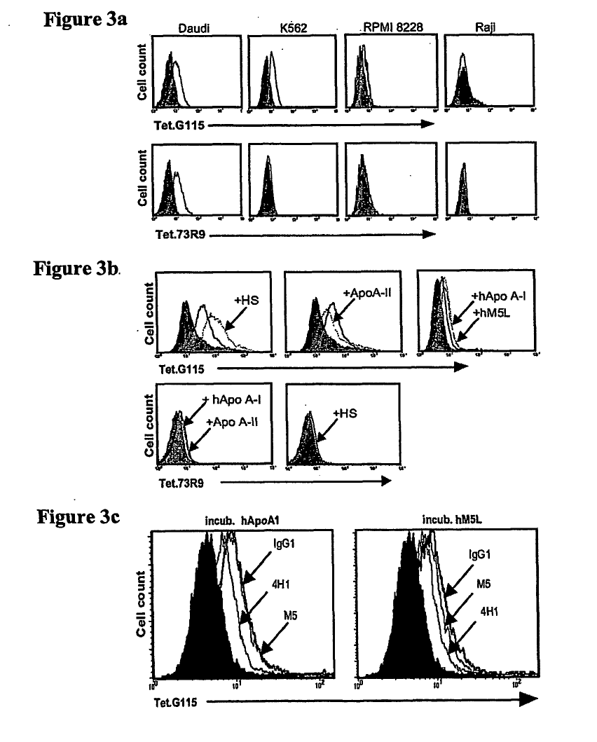

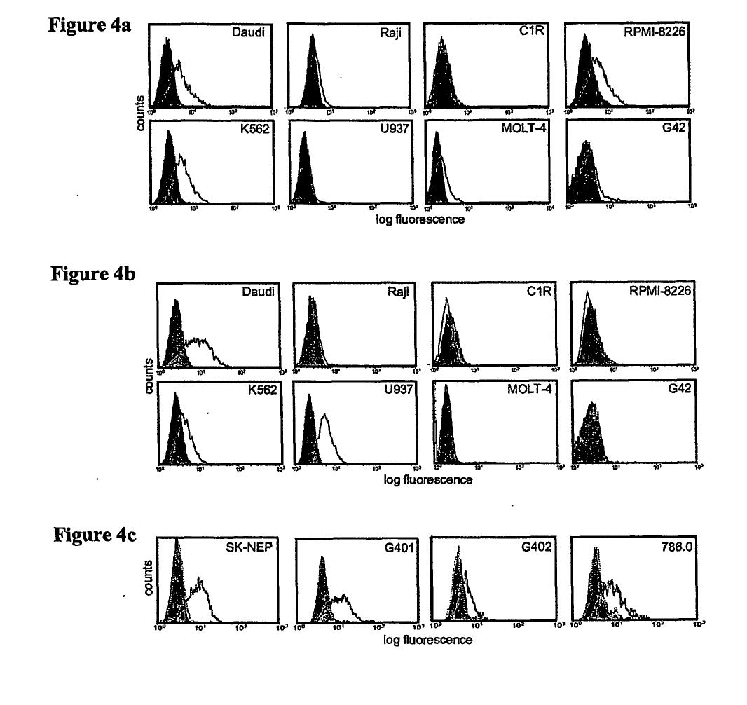

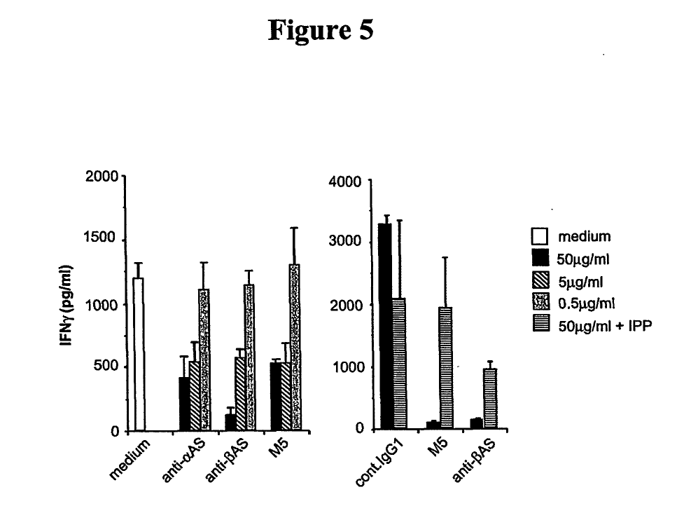

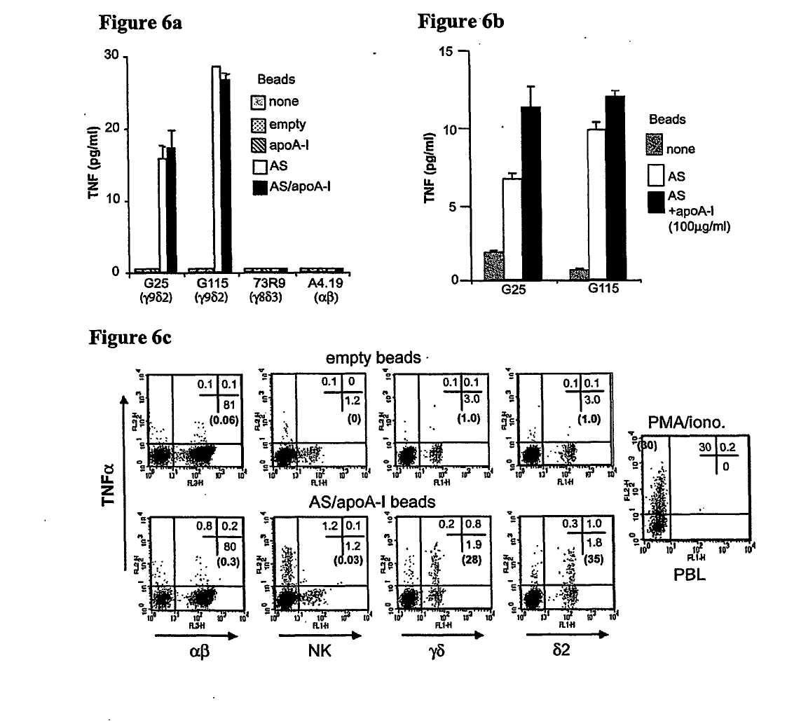

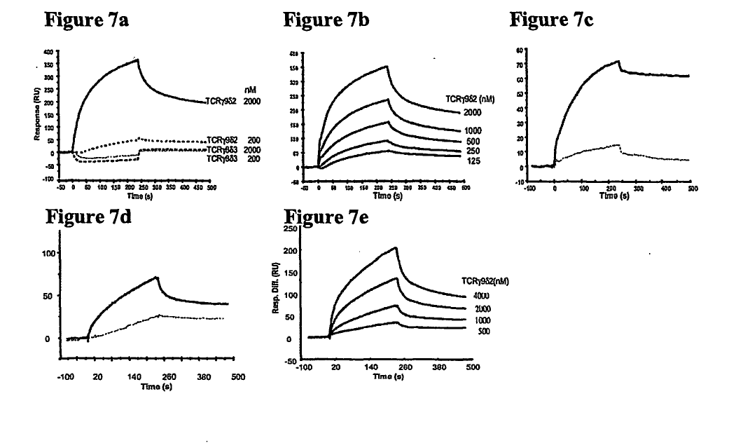

(b) Daudi cells were depleted of serum components by overnight culture in serum free medium (sfm) and subsequently incubated in PBS supplemented with either 10% fcs (sfm/fcs), 10% human serum (sfm/hs) or 1% BSA plus 100 μg/ml of M5L (sfm/M5L) before staining as in FIG. 1a.

(c) Serum-depleted cell lines were incubated with lipid-free HDL-apoA-I (solid lines and shaded histograms) or BSA (dotted lines) before indirect staining with the anti-human apoA-I antibody 4H1 (lines) or control IgG1 (shaded histograms).

(d) Left: coomassie-blue staining of M5-immunopurified material from fcs (lane 1) and human serum (lane 2) after non-reducing SDS-polyacrylamide gel electrophoresis (PAGE). Right: Immunoblot after reducing PAGE using anti-human apoA-I antibody 4H1 on: human serum (lane 3); fetal calf serum (lane 4); HDL (lane 5); hM5L human serum ligand of M5 mAb, lane 6).

(e): ¹²⁵I-labelled free apoA-I binding was measured after 2 hours incubation at 4° C. on Daudi and Raji cells. Top and bottom panels: binding isotherm of ¹²⁵I-labeled free apoA-I to Daudi (top) and Raji (bottom) cells. (◯): total binding, (σ): non-specific binding; (●): specific binding. Middle panel: Scatchard representation of specific binding on Daudi cells. In absence of specific binding on Raji cells, Scatchard representation could only be performed from Daudi cells data The results are representative of two different cell preparations.

FIG. 2: ApoA-I-dependent activation of Vγ9Vδ2 T cells. ⁵¹Cr release assays were performed in serum-free medium (sfm) to assess dependance on serum and apoA-I of Daudi cytolysis by different effector T cell populations. G25 and G42: Vγ9Vδ2 T cell clones, 73R9: Vγ8Vδ3 T cell clone. M5L (bovine serum component immunopurified with M5 antibody) was tested at the indicated concentration for its ability to substitute for serum (fcs). Recombinant annexin V was used as a control purified protein.

FIG. 3: Vγ9Vδ2 TCR binding to tumor cells and apoA-I. PE-labeled tetrameric TCRs from clone G115 and clone 73R9 were used to stain tumor cell lines and assess the influence of apolipoproteins on their tumor recognition. Shaded histograms: concentration-matched streptavidin-PE alone. Line histograms: binding of the indicated PE-labelled tetrameric TCR. (a) Cell lines were cultured in 10% fcs before staining with tetramers. (b) K562 cells were serum-depleted, incubated with 10% human serum or the indicated apolipoprotein preparations (100 μg/ml) and stained with G115 or 73R9 TCR tetramers. (c) Serum-depleted K562 cells were incubated with 100 μg/ml of HDL hApoA-I (left histogram) or hM5L (right histogram) and labeled with G115 TCR tetramers in the presence of the indicated competing mAb (20 μg/ml). Note that M5 does not recognize HDL hApoA-I (see text) whereas 4H1 recognizes both forms of the human protein. Data are representative of three experiments.

FIG. 4: Expression of ATP-synthase-related structures on tumor cells.

(a,b) Indirect immunofluorescence surface staining of haematopoietic tumor lines with (a) anti α-AS and (b) anti-β-AS. (c) Four kidney tumours sensitive to γ9δ2 lysis were tested for expression of AS by facs staining using control IgG (shaded histogram), anti-α-AS (dark line) and anti-β-AS (dotted line).

FIG. 5: ApoA-I and AS-dependent activation of Vγ9Vδ2 T cells. Daudi cells were incubated in serum, washed and incubated with serum-depleted Vγ9Vδ2 cells (clone G42) in sfm medium, in the presence of the indicated concentration of antibodies, and the production of γ-interferon was measured after a 20 hours co-culture. NaN₃-containing anti-AS and control antibodies were dialyzed before use (right panel). The phosphoantigen isopentenylpyrophosphate (IPP) was added in control cultures (2 μg/ml) to exclude a possible toxicity of M5 and anti-βAS antibodies (left panel).

FIG. 6: Induction of Vγ9Vδ2 T cell lymphokine secretion by immobilized ATP-synthase. HDL-derived apoA-I and ATP synthase were immobilized on latex beads and these were used to stimulate T cell populations. none: no stimulation; empty: beads saturated with BSA; apoA-I: beads coated with apoA-I only. AS: beads coated with the F1 extra-membrane subunit of bovine ATP synthase. AS/apoA-I: beads coated with both protein preparations.

(a) T cell clones were activated with protein-coated beads in medium supplemented with human serum, and TNFα secretion was measured in the culture supernatant after 4 hours. The capacity of γδ clones G25, G115, 73R9 to secrete significant amounts of TNFα upon stimulation was checked by using Daudi cells as target cells as well as PMA/ionomycin or PHA. The αβ clone A4.19 secretes saturating quantities of TNFα upon PMA/ionomycin or PHA stimulation (not shown).

(b) Stimulation was performed in the absence of exogenous serum (sfm) and purified HDL-derived apoA-I was added in some cultures (black bars).

(c) Fresh PBL were activated with indicated beads for 4 hours in the presence brefeldin A. Cells were then stained with antibodies to lymphocyte subsets, fixed, permeabilized and TNFα accumulation was analyzed after intracellular staining by flow cytometry. PMA/ionomycin stimulation is used as a control for TNFα-producing cells in the total population. Percentages indicating TNFα-producing cells within analysis quadrants and within each particular subset (between brackets) are shown.

FIG. 7. Surface plasmon resonance analysis. Soluble proteins were exposed to the sensorchip surface for 240 s (association phase) followed by a 240-s flow running (dissociation phase). Immobilized proteins were on sensorchips flowcell 2. Sensorgrams are representative of specific interactions (differential response) where non-specific binding that occurred on flow cell 1 (with no protein immobilized) was deduced from binding that occurred on flow cell 2. Results are expressed as resonance units (RU) as a function of time in seconds.

(a,b) Overlay sensorgrams for SPR analysis of soluble TCRγδ protein binding to immobilized apoA-I. Amount of immobilized ApoA-I protein was 2260 RU on flow cell 2 (a): comparative sensorgrams of soluble Vγ9Vδ2TCR and Vγ8Vδ3TCR (200 and 2000 nM) binding onto immobilized apoA-I. (b) Vγ9Vδ2TCR was injected at concentrations ranging from 125 nM to 2 μM. The apparent kinetic constants of the interaction were kₐ=8.8e³±6.08 e² M⁻¹, k_{d}=7.13 e⁻³±6.76 e⁻⁵s⁻¹, K_{D}=8.1 e⁻⁷M.

(c) Comparative sensorgrams of purified apoA-I (full line) and apoA-II (dotted line) binding to immobilized Vγ9Vδ2TCR (proteins were injected at 100 μg/ml). Amount of immobilized TCRγδ protein was 335 RU on flow cell 2.

(d,e) Comparative sensorgrams of soluble TCR binding to immobilized ATP synthase. Amounts of immobilized ATP synthase (F1) was 22440 RU on flow cell 2. (d) Comparative sensorgrams of purified monomeric G115 TCR (Vγ9Vδ2, full line) and 73R9 TCR (Vγ8Vδ3, dotted line) binding to immobilized ATP synthase (F1). Proteins were injected at a concentration of 2 μM. (e) Monomeric G115 soluble TCR (Vγ9Vδ2) was injected at concentrations ranging from 0.5 to 4 μM. The apparent kinetic constants of the interaction were kₐ=1.68 e³±2.16 M⁻¹s⁻¹, k_{d}=2.54 e⁻³±2.9 e⁻⁵s⁻¹, K_{D}=1.51 e⁻⁶M.

### DETAILED DESCRIPTION OF THE INVENTION

T cells of the γδ type are expressed by most mammalian species. They represent 1-10% of total circulating lymphocytes in healthy adult human subjects and most non-human primates. Most human peripheral blood γδ T cells express a γδTCR heterodimer encoded by Vγ9/Vδ2 genes, some NK-lineage receptors for MHC class I and almost no CD4 nor CD8. These cells have been shown to exhibit strong, non MHC-restricted, cytolytic activity against virus-infected cells (Poccia et al, J. Leukocyte Biology, 62, 1997, p. 1-5), parasite-infected cells (Constant et al, Infection and Immunity, vol. 63, n° 12, December 1995, p. 4628-4633), or tumor cells (Fournie et Bonneville, Res. Immunol., 66^{th} FORUM IN IMMUNOLOGY, 147, p. 338-347). These cells are also physiologically amplified in the context of several unrelated infectious diseases such as tuberculosis, malaria, tularemia, colibacillosis and also by B-cell tumors (for review see Hayday, 2000). These cells are thus viewed as potent effectors of innate immunity and represent an important resource of anti-infectious and anti-tumoral effectors. In this regard, γδT cell-mediated therapeutic programs are currently under development, particularly for treating cancers, using ex vivo activated cells or direct in vivo injection of γδT activators. However, in order to increase the efficacy of these treatments, it would be highly valuable to have methods available for selecting best responding patients or for selecting target cells, as well as for increasing the sensitivity of target or pathologic cells to these γδT cell-mediated therapies.

The instant invention now provides such methods.

As disclosed above, the invention relates to methods of assessing the sensitivity of cells to the activity of γδT cells, e.g., to their lysis or cytotoxicity. The tested cell may be any cell, preferably a mammalian cell, typically a human cell. The tested cell is generally a pathologic cell (i.e., a cell associated with or resulting from a disease in a subject), and the method is conducted in order to evaluate the efficacy of a γδT cell-mediated therapy against said pathology.

Typical examples of such target or test cells include pathogenic mammalian (e.g., human) tumor cells, pathogen-infected cells, pathologic immune cells, etc. Specific examples of tumor cells include any solid or hematopoietic tumor cell, such as renal cancer cells, liver cancer cells, lung cancer cells, bladder cancer cells, breast cancer cells, colon cancer cells, CNS cancer cells, head-and-neck cancer cells, etc. The sample used may be any isolated pathologic cell, or a tissue, organ, tissue sample, body fluid, etc. In a specific embodiment, the method is carried out on a biopsy from a subject, typically a biopsy section. The method may be performed on a sample collected from a subject and stored, optionally in frozen state, or extemporaneously, upon collection from the subject.

Determination of responsiveness or sensitivity to γδT cell comprises, within the context of the invention, an assessment of cell surface expression of an ATP synthase polypeptide or a fragment thereof. Indeed, the present invention now shows that ATP synthase participates in γδT cell activation, and is directly involved in TCR recognition and binding. The invention also shows that cells that are sensitive to γδT cells express at their surface an ATP synthase, while such protein is normally expressed intracellularly (i.e., essentially in mitochondria). By detecting ATP synthase cell surface expression, sensitivity of a target cell to γδT cells may thus be evaluated.

Within the context of this invention, an ATP synthase polypeptide designates any polypeptide, protein or peptide fragment of an ATP synthase, as well as variants, isoforms and orthologs thereof. The sequence of ATP synthase genes and proteins has been disclosed in the prior art literature, and is available in various gene libraries. In particular, the amino acid sequence of a human ATP synthase protein is available on SwissProt Data base under the following accession numbers: F1-α subunit: P25705; F1-β subunit: PO6576; F1-γ subunit: P36542; F1-δ subunit: 30049; F1-ε subunit: P56381. The nucleotide sequence of a human ATP synthase cDNA is available on GenBank Data base under the following accession numbers: F1-α subunit: BT007209; F1-β subunit: NM01686; F1-γ subunit: XM292254; F1-δ subunit: NM001687; F1-ε subunit: BT007293. Further information regarding the cloning, sequence, characteristics, expression and structure-function of a human ATP synthase gene or protein can be found in Kataoka, H. and Biswas, C., Biochim. Biophys. Acta 1089 (3), 393-395 (1991); Neckelmann, N., et al., Genomics 5 (4), 829-843 (1989); Matsuda, C., et al., J. Biol. Chem. 268 (33), 24950-24958 (1993) and Biochim. Biophys. Acta 1130 (1), 123-126 (1992) or Tu, Q., et al., Biochem. J. 347 Pt 1, 17-21 (2000), incorporated therein by reference.

The mitochondrial ATP synthase is typically composed of an intramembrane sector termed F0 and an extramembrane sector termed F1. The F1 sector comprises α, β, γ, δ and ε subunits in an α3β3γδε polypeptidic complex. At least the α and β polypeptides show ectopic expression and can be found at the surface of pathologic cells, such as tumor cells.

Accordingly, in a specific embodiment, the ATP synthase polypeptide is an ATP synthase α subunit.

In an other specific embodiment, the ATP synthase polypeptide is an ATP synthase β subunit

In a further specific embodiment, the ATP synthase polypeptide is a complex formed between an ATP synthase α and β subunits.

In an other specific embodiment, the method comprises detecting a portion of an ATP synthase, typically an extracellular domain of an ATP synthase α or β subunit.

Determination of said cell surface expression can be performed using a variety of techniques known per se, such as by ligand binding, immunohistochemistry, etc. In a specific embodiment, the method comprises incubating the biological sample comprising said test, target or pathogenic cells (e.g., cultured cells, tissue or organ) with a ligand specific for an ATP synthase polypeptide or a portion thereof, and assessing binding of said ligand to said cell, tissue or organ. In a more preferred embodiment, the ligand is an antibody or a fragment or derivative thereof (e.g., a Fab or Fab′2 fragment, a CDR region, a SCfV, etc.). The ligand or antibody may be labelled, e.g., by fluorescent, enzymatic, luminescent, radioisotope, etc. labels, to facilitate binding determination.

The ATP synthase polypeptide, particularly the extracellular domains thereof, may be detected by indirect immnunofluorescence using commercially available antibodies. As a specific example, antibodies specific for the α or β subunits may be found at Molecular Probes, Inc., under the following references:
anti ATP synthase subunit alpha: clone 7H10 (product A-21350)
anti ATP synthase subunit beta: clone 3D5 (product A-21351)

In a specific embodiment, the method comprises providing a biopsy from the patient and determining cell surface expression of an ATP synthase polypeptide or a portion thereof by immunohistochemistry using an antibody.

As mentioned, the method allows the design of improved or customized therapies, by determining which patients would best respond to particular γδT cell-mediated therapies. In this regard, the γδT cell-mediated therapy may be a direct in vivo therapy (e.g., comprising the injection of a drug that stimulates γδT cells in said patient) or an ex vivo cell therapy (e.g., comprising the injection of ex vivo activated γδT cells to said patient).

The invention also provides novel methods of stimulating γδT cells, comprising contacting γδT cells with an ATP synthase polypeptide or a TCR-binding fragment thereof. Alternatively, the invention also provides methods of stimulating γδT cells comprising contacting γδT cells with an apolipoprotein A1 polypeptide or a variant or ATP synthase—or TCR-binding fragment thereof. The present invention indeed discloses that Apolipoprotein A-I is a ligand of ATP synthase and of the TCR of lymphocytes Tγ9δ2, so that this polypeptide or, more preferably, derivatives thereof, represent valuable therapeutic agents to mediate γδT cell therapy. These derivatives are preferably designed to have an increased affinity for the cell surface-expressed ATP synthase and/or for the TCRγ9δ2, allowing the specific targeting of this lymphocyte population toward pathologic (e.g., tumor) cells.

These stimulation methods can be carried out in vivo, in vitro or ex vivo.

In a specific embodiment, the method comprises contacting γδT cells with a complex comprising an ATP synthase polypeptide or a TCR-binding fragment thereof linked to an apolipoprotein A1 polypeptide. In a further preferred embodiment, the ATP synthase polypeptide or fragment is immobilized on a support. Indeed, as shown in the examples, solid support coated with ATP synthase polypeptide is able to stimulate γδT cells. The support may be a bead, column, plate, etc. The γδT cells are preferably human cells.

In this respect an object of this invention resides in a product comprising, immobilized on a support, an ATP synthase polypeptide or a TCR-binding fragment thereof.

A further object of this invention resides in a method of increasing sensitivity of a target cell to γδT cell lysis or cytotoxicity, comprising causing or increasing cell surface expression of an ATP synthase polypeptide or a portion thereof in said target cell.

A further object of this invention is a method of screening, selecting or identifying a compound, comprising:
a) separately contacting γδT cells with an ATP synthase polypeptide or a TCR-binding fragment thereof in the presence and in the absence of a candidate compound and,
b) screening, selecting or identifying a compound that modulates activation of said γδT cells by said ATP synthase polypeptide or a TCR-binding fragment thereof.

The method is typically conducted in vitro, in any suitable device, such as a plate, tube, flask, pouch, etc. The method is particularly suited for use in multi-well plates, to screen several compounds in parallel. In the above screening methods, the ATP synthase polypeptide or TCR-binding fragment thereof may be in suspension or immobilized on a support, such as a bead or at the surface of the device itself. Also, the method may be conducted in the presence of an apolipoprotein A1 polypeptide. Furthermore, the method is suitable for selecting or designing or improving apolipoprotein A1 polypeptide variants that modulate activation of γδT cells. The activity of γδT cells may be assessed using a number of biological assays, such as proliferation, target cell lysis, cytokine release, etc. The method is particularly suited to identify compounds that activate γδT cells.

Further aspects and advantages of this invention will be disclosed in the following experimental section, which should be regarded as illustrative and not limiting the scope of this application.

### MATERIAL AND METHODS

1) Tumor Cell Lines, T Cell Clones and Cultures

Daudi, Raji, RPMI 8226, K562, Jurkat, Molt-4, and U937, SK-NEP, G401, G402, and 786.0 were obtained from ATCC. Awells (EBV⁺ lymphoblastoid B cell line) is from the International Histocompatibility Workshop (IHW#9090). C1R (HLA-A⁻B⁻-LCL) and RPMI 8866 (B-LCL) were obtained from Drs P. Lebouteiller and M. Colonna respectively. All tumor cell lines were cultivated in RPMI 1640 medium supplemented with 10% fetal calf serum (Invitrogen) except for serum deprivation experiments. In this case, cells were washed once in RPMI 1640, incubated for 1 hour at 37° C. in serum free culture medium (hybridoma sfm, Invitrogen), pelletted and reincubated for at least 16 hours at 37° C. in sfm before use. The G25 (Vγ9Vδ2) and 73R9 (Vγ8Vδ3) clones were obtained as described for G42 and G115 (Allison et al., 2001; Davodeau et al., 1993) by anti-Vδ monoclonal antibody selection and subsequent amplification using PHA and IL2 and cloning.

3) Fluorescence Analysis and Antibodies

Immunofluorescence stainings were performed in PBS containing 1% BSA and devoid of serum using FITC-conjugated goat F(ab)′₂ anti-mouse Ig antibody (Caltag) as the second step reagent. Irrelevant isotype-matched control antibodies were used as negative controls. In apolipoprotein binding experiments, serum-deprived cells were incubated with serum or purified protein preparations in PBS containing 1% BSA, at room temperature, 30 minutes prior to antibody staining. 7H10 (anti-α-ATP synthase) and 3D5 (anti-β-ATP synthase) and 7F9 (anti-γ-ATP synthase) are from Molecular Probes. 4H1 (anti-human apoA-I) (Collet et al., 1997) was obtained from Dr Y. Marcel (Ottawa).

4) Generation of M5A12D10 Hybridoma and Antibody Purification

Balb/c mice were injected intraperitoneally four times at two-weeks intervals with 15×10⁶ Daudi cells washed and resuspended in PBS. Hybridoma were obtained by fusion of spleen cells with P3X63Ag8 myeloma cells and were selected on the basis of tumor cell staining. Subcloned hybridoma were subsequently amplified in sfm medium and antibodies were purified on protein G affinity columns (Pharmacia), neutralized, dialyzed against PBS and concentrated (Harlow and Lane, 1988). Finally, antibodies were tested for their ability to modulate lysis of Daudi cells by γδ effectors, leading to the selection of the M5A12D10 antibody (IgG1).

5) Purified Proteins

The human and bovine ligands of M5A12D10 (hM5L and hM5L respectively) were isolated by affinity chromatography: human and fetal calf serum diluted 1/20 in 3 M NaCl and 50 mM Tris pH 7 were passed through the column carrying the covalently attached antibody. After washing (last wash was in 3 M NaCl, 10 mM Tris pH 7), bound proteins were eluted with 100 mM glycine pH 2.7. Isolation of ApoA-I from High Density Lipoproteins (HDL apoA-I) by ion-exchange chromatography has been already described (Mezdour et al., 1987). Purity of apoA-I was checked by Western blot analyses using different antibodies directed against human apoB, apoA-II, apo-C and apoA-I. The apolipoprotein A-I homogeneity was more than 99% (as measured by densitometry after SDS-PAGE and silver staining). Purified bovine ATP synthase (F1 subunit) (Lutter et al., 1993) was obtained from John E. Walker (Cambridge, UK).

6) Production of Soluble Fluorescent Tetrameric TCR and Usage for Cell Staining

The G115 (Vγ9Vδ2) (3) and 73R9 (Vγ8Vδ3) extracellular γ and δ chains (the latter carrying a short 3′ Biotin tag) were expressed in Escherichia coli, then refolded together by rapid dilution in 1 L of 1 M L-arginine, 0.1 M Tris-HCl, pH 8.0 and 0.2 mM reduced/0.2 mM oxidized glutathione. After dialysis against 10 mM Tris-HCl pH 8.0, concentration by cation exchange chromatography at pH 5.5, and purification by size exclusion chromatography at pH 8.0, the refolded protein was biotinylated for 4 h at 30° C. with 6 μg/ml BirA and excess of free biotin was removed by dialysis against 10 mM Tris-HCl pH 8.0, 150 mM NaCl. Tetramers of γδ TCR heterodimers were obtained by mixing the biotinylated TCR with phycoerythrin-labeled streptavidin (Biosource) at a molar ratio of 10:1. For staining, 2.10⁵ cells were incubated at room temperature for 45 min with phycoerythrin-labeled γδ TCR tetramers at a concentration of TCR of 30 μg/ml in PBS plus 1% BSA. Background staining was determined using phycoerytlrin-labeled streptavidin at the same concentration.

7) Protein Identification by Proteomic Analysis and Mass Spectrometry

Immunopurified material was analyzed using one dimensional electrophoresis and visualized by coomassie blue staining. Protein bands were excised from the gel and subjected to several washing steps, reduction alkylation reaction, in-gel trypsin digestion with modified trypsine (Promega, Madison, Wis.) at 25 ng/μl in 50 mM NH₄HCO₃ and finally followed by peptide extraction. The peptides purified with ZipTip C18 (Millipore) were mixed with equal volumes (0.5 ml) of a saturated a-cyano-4-hydroxycinnamic acid in 50% acetonitrile, 0.1% TFA onto the MALDI target and allowed to air-dry. Peptide mass fingerprinting were obtained by using a PE Biosystems MALDI-TOF mass spectrometer (Voyager DE STR, Foster City, Calif., USA) on each protein band. Unknown proteins were identified using the data base fitting program MS-Fit (Protein Prospector, (http://prospector.ucsf.edu)), searching against all eukaryotic entries in Swiss Prot and NCBI non redundant protein data bases. We considered the identifcation positive when a minimum of four measured peptide masses were matched and provided at least around 20% sequence coverage. Mass accuracy of 10 ppm was obtained with internal calibration using auto-digestion peaks of trypsin (M+H+, 842.51, 2211.10, and 2283.18).

8) Chromium and Cytokine Release Assays

2 h- ⁵¹Cr-release assays were performed in standard conditions except for the use of serum-free conditions in some experiments: in sensitization experiments with apolipoprotein preparations, target tumor cells were serum-deprived as described for facs analysis, loaded with ⁵¹Cr (100 μCi/10⁶ cells, 1 hour, 37° C.), extensively washed in RPMI and resuspended in sfm medium. Effector T cells cultivated in serum-containing medium were washed extensively in RPMI, incubated for 2 hours in sfm at 37° C. and resuspended in sfm medium. Target cells (3000/well, in triplicates) were first incubated with apolipoprotein preparations or serum-containing medium for 30 min at room temperature in 96-well round-bottom microculture plates at room temperature prior to the addition of effector cells. Cells were then pelleted and incubated at 37° C. for 2 hours. Supernatants were recovered for ⁵¹Cr release measurement. Spontaneous release (in the absence of effectors) was subtracted from experimental data and was in the 10-30% range of maximum release (effector cells replaced by same volume of 0.1 M HCl). Specific lysis was calculated as the percentage of maximum release. For cytokine release measurements, similar experiments were performed supernatants were harvested after 4-hours (TNFα) or 20 hours (IFNγ). IFNγ was titrated by a specific Elisa technique, whereas TNFα concentration was assessed by a biological assay based on WEHI cells viability.

9) Binding Assays

The binding experiments were performed at 4° C. for 2 hours as previously described (Barbaras et al., 1994). Briefly, cells (9 μg of cell proteins per point) were incubated in PBS for 2 hours at 4° C. with increasing concentrations of labeled apoA-I. Cells were filtered on 0.22 μm filters (GVWP Millipore-France) and washed four times with 1% BSA in PBS. Filters were used for radioactivity measurements. Non-specific binding was determined in the presence of a 100-fold excess (as compared to the K_{D} value) of the corresponding unlabeled ligand. Binding was analyzed using a weighted non-linear curve-fitting program, based on the LIGAND analysis program (Prism-GraphPad).

10) Immobilization of Proteins on Latex Beads.

10⁷ sulfate latex beads (Interfacial Dynamics corp., Portland, Oreg.) were washed in PBS and incubated with apolipoproteins (100 μg/ml), F1-ATP synthase (0.4 mg/ml) or a mixture of both under constant agitation at room temperature for 16 hours. Beads were then washed, saturated for 3 hours in PBS containing 1% BSA and washed extensively in PBS before use. Control “empty” beads were similarly saturated with BSA. In stimulation experiments, beads were mixed with cells at a 1:1 ratio.

11) Immobilization of Proteins for SPR (Biacore) Analysis.

ApoA-I and ATP synthase (F1 subunit) were immobilized by amine linkage on CM5 chips (Biacore AB) following NHS-EDC activation; Vγ9Vδ2-biotinylated soluble TCRs were immobilized on SA streptavidin-coated chips (Biacore AB) and binding was analyzed in a Biacore 3000 apparatus (BiacoreAB). Soluble ligands were injected at a flow rate of 20 μl/min, exposed to the surface for 240 s (association phase) followed by a 240-s flow running during which the dissociation occurred. Sensorgrams are representative of specific interactions (differential response) where non-specific binding that occurred on flow cell 1 was deduced from binding that occurred on flow cell 2. Results are expressed as resonance units (RU) as a function of time in seconds.

Results

In an attempt to characterize such antigens, we raised murine monoclonal antibodies against the Burkitt's lymphoma Daudi, a cell line which is lysed by a large majority of Vγ9Vδ2 T cell clones (Davodeau et al., 1993; De Libero et al., 1991). We selected monoclonal antibodies (mAb) for their differential binding to Daudi and Raji (a non-activating Burkitt's lymphoma) and for their ability to interfere with Vγ9Vδ2 T cell recognition of Daudi cells. One mAb (#M5A12D10, hereafter referred to as M5) which fulfilled both criteria was selected for further studies. M5 mAb stained Daudi cells as well as several other hematopoietic tumors reported to be recognized by Vγ9Vδ2 T cells, including cell lines of lymphoid (MOLT-4, RPMI 8226) and myeloid (K562, U937) origins. By contrast several tumor cells resistant to Vγ9Vδ2 T cell killing such as Raji and B lymphoblastoid cells were not stained by M5 mAb (FIG. 1a). As M5 mAb binding to tumor cells was dependent on the presence of serum in cell culture medium (FIG. 1b), M5 was subsequently used to immunopurify a putative ligand from bovine serum. A protein (M5L) running in polyacrylamide gels as a ˜28 kDa polypeptide could be isolated and mass spectrometry analysis of tryptic peptide digests identified apolipolipoprotein A-I as the likely ligand. M5 immunopurified a similar protein from human serum (hM5L) and its identity to apoA-I was confirmed by its reactivity in immunoblotting experiments using the anti-human apoA-I monoclonal antibody 4H1 (FIG. 1d). Accordingly, like M5 Ab, 4H1 stained Daudi and RPMI 8226, another tumor line frequently killed by Vγ9Vδ2 T cells, but not Raji cells following their incubation with a human lipid-free form of human apoA-I prepared from high density lipoprotein particles (HDL-apoA-I) by ion exchange chromatography (FIG. 1c). In order to assess the putative involvement of apo A-I in Vγ9Vδ2 cytolytic activity, we performed cytotoxicity assays in serum-free medium (sfm), using target cells depleted of serum after an overnight culture in sfm (FIG. 2). Cytolytic activity of Vγ9Vδ2 T cell clones against Daudi cells was decreased in serum-free conditions, and restored by addition of serum during the cytotoxicity assay. Similarly, addition of purified M5L increased Vγ9Vδ2 T cell-mediated lysis in a dose-dependent manner. By contrast Daudi cell lysis by a control Vγ8Vδ3 T cell clone (#73R9) was neither affected by serum deprivation nor by addition of M5L (FIG. 2). A similar effect on Vγ9Vδ2 T cell cytotoxicity was observed using HDL-apoA-I or a commercial apoA-I preparation (Sigma, data not shown). Thus, tumor lysis by Vγ9Vδ2 T cells is specifically affected by soluble extracellular apoA-I.

Modulation of Vγ9Vδ2 T cell activity by apo A-I could be due to apoA-I recognition by either the Vγ9Vδ2 TCR itself or by accessory receptors such as toll-like receptors which are known to be expressed on various conventional and non-conventional T cell subsets (Caramalho et al., 2003; Mokuno et al., 2000; Sakaguchi, 2003). Possible involvement of apoA-I in TCR engagement was studied by using recombinant soluble forms of the Vγ9Vδ2 TCR derived from the G115 clone (Allison et al., 2001), which were biotinylated and tetramerized by fluorescent streptavidin (hereafter referred to as TCR tetramers). Like M5 mAb, Vγ9Vδ2 TCR tetramers bound to the Vγ9Vδ2 target cells Daudi, K562 and more weakly to RPMI 8226, but neither to Raji cells nor to B lymphoblastoid cell lines (FIG. 3a and data not shown). As a control, Vγ8Vδ3 TCR tetramers derived from clone 73R9 bound to Daudi but to a much lesser extent to K562 cells (FIG. 3a) indicating that these two TCRs target distinct structures on the tumor cell surface. K562 cells, which yielded the brightest staining levels with Vγ9Vδ2 TCR tetramers, were used in further experiments. Vγ9Vδ2 TCR tetramer binding to K562 was decreased when cells were cultured for 18 h in the absence of serum (not shown) and increased following addition of serum or human apoA-I (hM5L or HDL-apoA-I). Importantly apoA-II, whose hydrophobicity was similar to that of apoA-I, did not enhance Vγ9Vδ2-TCR tetramer binding (FIG. 3b). This suggested that the effect of apoA-I was not merely due to a non-specific adsorption of TCR tetramers, which might have been induced by hydrophobic compounds. Accordingly neither serum, nor apoA-I, affected the binding of Vγ8Vδ3 TCR tetramers (FIG. 3b). Also consistent with a specific effect of apoA-I on Vγ9Vδ2 TCR tetramer binding, pre-incubation of cells with M5 and 4H1 antibodies decreased TCR tetramer binding to cells coated with hM5L. 4H1 decreased tetramer binding on cells preincubated with HDL-derived apoA-I whereas M5 did not (FIG. 3c). This goes along with the observation that M5 mAb does not bind to HDL-derived apoA-I (not shown) and suggests structural differences between the immuno-purified and the HDL-derived forms of apoA-I, although both are recognized by 4H1 and promote TCR binding. Although soluble Vγ9δ2 TCR no longer bound to serum-deprived Daudi cells (data not shown), they still stained serum-deprived K562 cells (FIG. 3b). This could be explained by the persistence of residual apolipoproteins on the cell surface. However, it remained possible that the soluble TCR bound to an as yet unidentified structure and that this binding was increased by apoA-I.

We have recently shown that components of the mitochondrial enzyme ATP-synthase (AS) can be expressed on the surface of hepatocytes, and that this enzyme constitutes a high affinity receptor for free, delipidated apoA-I on these cells (Martinez et al., 2003). We studied expression of components of this enzyme on the surface of Vγ9Vδ2 tumor targets. Daudi, K562 and RPMI 8226 were stained by mAb against the a chain of AS (αAS) whereas Raji, leukaemic T cells and B-LCL were not (FIG. 4a). Four kidney tumors which were lysed by Vγ9Vδ2 CTL also expressed an αAS-related surface component (FIG. 4c). The β subunit of AS was also detected on Daudi, K562 and U932, a monocytic line not consistently killed by Vγ9Vδ2 T cells. This chain was undetectable on RPMI 8226 and the kidney tumors (FIG. 4b,c). Therefore tumor susceptibility to Vγ9Vδ2 lysis more strongly correlated with expression of the αAS subunit. Scatchard analysis of the binding of iodinated apoA-I to Daudi cells revealed a single binding receptor on these cells (K_{D}=0.8×10⁻⁷M; FIG. 1e). Since Daudi cells did not express other putative apoA-I receptors, such as Scavenger Receptor B1 (Murao et al., 1997) or ABCA-1 (Chambenoit et al., 2001) (data not shown), these experiments strongly suggested that AS was the apoA-I receptor on tumor cells.

Two sets of experiments were performed to assess the involvement of AS in tumor cell recognition by Vγ9Vδ2 T cells. Firstly, antibodies to apoA-I and to the extramembrane (F1) subunit of AS were tested for their ability to modulate lymphokine secretion by Vγ9Vδ2 T cells following incubation with Daudi cells. IFNγ secretion was strongly inhibited by anti-αAS, anti-βAS and M5, as compared to a control Ig. This inhibition was not merely due to Ab toxicity since Vγ9Vδ2 T activation was largely restored by addition of isopentenyl pyrophosphate (IPP), a previously described Vγ9Vδ2 T cell antigen (FIG. 5).

Secondly, the involvement of AS in Vγ9Vδ2 T cell activation was studied by testing the ability of mitochondrial bovine AS immobilized on latex beads with and without apoA-I to stimulate Vγ9Vδ2 T cells. Beads carrying apoA-I only or none of these proteins were also tested (FIG. 6). Co-culture with AS-coated beads induced a strong TNFα secretion by Vγ9Vδ2 clones, whereas Vγ8Vδ3 and αβ clones, otherwise able to produce TNFα after stimulation by phorbol myristate acetate and calcium ionophore, were not activated (FIG. 6a). We could not demonstrate any stimulatory activity of immobilized apoA-I alone. Similar experiments performed in serum-free conditions indicated a stimulatory activity of beads carrying AS alone, and a relatively minor increase of this stimulatory activity following addition of soluble apoA-I (FIG. 6b). This would suggest an accessory role of apoA-I in the stimulatory activity of AS. However a possible carryover of apoA-I or related apolipoproteins by effector cells cannot be ruled out. When added onto fresh PBL, beads carrying AS and apoA-I induced selective TNFα production by a large fraction of Vδ2⁺ T cells but had no significant effect on the αβ and NK cell populations (FIG. 6c).

Altogether the above results suggested a highly specific and TCR-mediated activation of Vγ9Vδ2 cells by apoA-I and AS. However, the respective contribution of AS and apoA-I in γδ T cell/tumor cell interactions remained unclear. Although anti-apoA-I antibodies had a substantial inhibitory effect on cytotoxicity and lymphokine secretion, these effects as well as the modulatory properties of apoA-I could be explained by the proximity of apoA-I and AS on the cell surface, possibly generating indirect perturbation of TCR-AS interactions. Surface plasmon resonance analysis (SPR) has been previously used to demonstrate interactions between apoA-I and ATP synthase (Martinez et al., 2003). We followed a similar approach to assess TCR/apoA-I/AS interactions, after immobilization of either apoA-I, soluble G115 TCR or purified AS onto SPR chips. Immobilized apoA-I specifically interacted with soluble monomeric G115 TCR (K_{D}~0.8 μM) whereas interaction with the 73R9 TCR was too low to be measured (FIG. 7a,b). Conversely, immobilized G115 TCR specifically interacted with soluble apoA-I, but not with apoA-II (FIG. 7c). When soluble TCR were similarly tested on immobilized AS, a clear interaction was demonstrated for the Vγ9Vδ2 TCR (K_{D}=1.5 μM) whereas the binding of the Vγ8Vδ3 TCR was low and could not be measured (FIG. 7d,e). Thus SPR analysis confirmed the occurrence of specific interactions between Vγ8Vδ2 TCR, AS and apo A-I.

Discussion

Our results strongly suggest that during interaction between Vγ9Vδ2 T cells and their tumoral targets, a ternary complex forms between the TCR on the T cell side and apoA-I plus AS on the tumor cell side. It is not clear whether the structure expressed on the surface of tumors is identical to mitochondrial AS: reactivity with available monoclonal antibodies suggests that the αAS and βAS chains can be independently expressed on various tumors, indicating some form of surface expression polymorphism of these proteins. Using a limited panel of tumor lines we found that sensitivity to Vγ9Vδ2 T cells correlated better with αAS than with βAS expression. Although AS might not be the primary tumor target antigen of Vγ9Vδ2 T cells but instead a cross-reactive unrelated entity, the stimulatory potential of immobilized mitochondrial AS would indicate that its homology to the putative primary antigen(s) is high enough to induce a potent Vγ9Vδ2 T cell stimulation.

Direct binding of apoA-I to AS (Martinez et al., 2003) and the TCR (our present data) have now been demonstrated. These interactions seem to be required for optimal T cell activation as Vγ9Vδ2 T cell activation and soluble TCR binding were markedly decreased by serum depletion and were restored by addition of exogenous soluble apoA-I. However, our present results suggest a hierarchical importance of these interactions for Vγ9Vδ2 T cell activation. Indeed, we could not demonstrate any stimulatory activity for immobilized apoA-I whereas AS appeared to be stimulatory by itself when immobilized on latex beads. SPR experiments however strongly suggest a direct interaction of apoA-I with both the TCR and AS. Thus a possible role for apoA-I could be the stabilisation of the interaction between the TCR and the AS, explaining the stimulatory effect of apoA-I in cytotoxicity assays.

ApoA-I is unlikely to be immunogenic by itself and, as already mentionned, immobilized apoA-I did not activate Vγ9Vδ2 T cells. However, apoA-I may serve as a carrier for antigenic ligands such as phosphoantigens, either derived from the tumor cells themselves or from the extracellular medium, thus permitting their “presentation” by a surface AS-related structure. This possibility would be consistent with the recently described correlation between endogenous production of mevalonate pathway metabolites (including IPP) by tumor cells and their susceptibility to Vγ9Vδ2 T cell-mediated lysis (Gober et al., 2003).

Ectopic expression of components of AS is not unprecedented as the presence of AS was previously described on the surface of K562 cells (Das et al., 1994) and endothelial cells (Moser et al., 1999) and linked to immunomodulatory effects in one case. The α/βAS subunits are also found on hepatocytes where they promote binding of free apoA-I and display enzymatic activity (Martinez et al., 2003). Whether these components are also involved in an enzymatic complex on tumor cells is not known yet. Although expression of this apoA-I receptor on normal tissue cells could potentially induce Vγ9Vδ2 T lymphocyte-mediated autoimmunity, several recent studies have described mechanisms involving NK-like receptors and permitting efficient control of this potential self reactivity (Fisch et al., 1997; Halary et al., 1997).

Besides, together with the high expression of LDL receptors and apoE in intraepithelial γδ lymphocytes (Fahrer et al., 2001), and with studies documenting apoE binding to ATP-synthase (Mahley et al., 1989), these data open a new field of investigations linking lipid metabolism and anti-tumor immunosurveillance.

### REFERENCES

Allison, T. J., Winter, C. C., Fournie, J. J., Bonneville, M., and Garboczi, D. N. (2001). Structure of a human gammadelta T-cell antigen receptor. Nature 411, 820-4.

Barbaras, R., Collet, X, Chap, H., and Perret, B. (1994). Specific binding of free apolipoprotein A-I to a high-affinity binding site on HepG2 cells: characterization of two high-density lipoprotein sites. Biochemistry 33, 2335-40.

Beckman, E. M., Porcelli, S. A., Morita, C. T., Behar, S. M., Furlong, S. T., and Brenner, M. B. (1994). Recognition of a lipid antigen by CD1-restricted alpha beta+ T cells [see comments]. Nature 372, 691-694.

Bukowski, J. F., Morita, C. T., and Brenner, M. B. (1999). Human gamma delta T cells recognize alkylamines derived from microbes, edible plants, and tea: implications for innate immunity. Immunity 11, 57-65.

Bukowski, J. F., Morita, C. T., and Brenner, M. B. (1994). Recognition and destruction of virus-infected cells by human gamma delta CTL. J. Immunol. 153, 5133-5140.

Bukowski, J. F., Morita, C. T., Tanaka, Y., Bloom, B. R., Brenner, M. B., and Band, H. (1995). V gamma 2V delta 2 TCR-dependent recognition of non-peptide antigens and Daudi cells analyzed by TCR gene transfer. J. Immunol. 154, 998-1006.

Caramalho, I., Lopes-Carvalho, T., Ostler, D., Zelenay, S., Haury, M., and Demengeot, J. (2003). Regulatory T cells selectively express toll-like receptors and are activated by lipopolysaccharide. J Exp Med 197, 403-11.

Chambenoit, O., Hamon, Y., Marguet, D., Rigneault, H., Rosseneu, M., and Chimini, G. (2001). Specific docking of apolipoprotein A-I at the cell surface requires a functional ABCA1 transporter. J Biol Chem 276, 9955-60.

Collet, X, Marcel, Y. L., Tremblay, N., Lazure, C., Milne, R. W., Perret, B., and Weech, P. K (1997). Evolution of mammalian apolipoprotein A-I and conservation of antigenicity: correlation with primary and secondary structure. J Lipid Res 38, 634-44.

Constant, P., Davodeau, F., Peyrat, M. A., Poquet, Y., Puzo, G., Bonneville, M., and Fournie, J. J. (1994). Stimulation of human gamma delta T cells by nonpeptidic mycobacterial ligands. Science 264, 267-70.

Das, B., Mondragon, M. O., Sadeghian, M., Hatcher, V. B., and Norin, A. J. (1994). A novel ligand in lymphocyte-mediated cytotoxicity: expression of the beta subunit of H+ transporting ATP synthase on the surface of tumor cell lines. J Exp Med 180, 273-81.

Davodeau, F., Peyrat, M. A., Hallet, M. M., Gaschet, J., Houde, I., Vivien, R., Vie, H., and Bonneville, M. (1993). Close correlation between Daudi and mycobacterial antigen recognition by human gamma delta T cells and expression of V9JPC1 gamma/V2DJC delta-encoded T cell receptors. J. Immunol. 151, 1214-1223.

Davodeau, F., Peyrat, M. A., Hallet, M. M., Houde, I., Vie, H., and Bonneville, M. (1993). Peripheral selection of antigen receptor junctional features in a major human gamma delta subset. Eur J Immunol 23, 804-8.

De Libero, G., Casorati, G., Giachino, C., Carbonara, C., Migone, N., Matzinger, P., and Lanzavecchia, A. (1991). Selection by two powerful antigens may account for the presence of the major population of human peripheral gamma/delta T cells. J. Exp. Med. 173, 1311-1322.

Fahrer, A. M., Konigshofer, Y., Kerr, E. M., Ghandour, G., Mack, D. H., Davis, M. M., and Chien, Y. H. (2001). Attributes of gammadelta intraepithelial lymphocytes as suggested by their transcriptional profile. Proc Natl Acad Sci USA 98, 10261-6.

Fisch, P., Meuer, E., Pende, D., Rothenfusser, S., Viale, O., Kock, S., Ferrone, S., Fradelizi, D., Klein, G., Moretta, L., Rammensee, H. G., Boon, T., Coulie, P., and Vanderbruggen, P. (1997). Control Of B Cell Lymphoma Recognition Via Natural Killer Inhibitory Receptors Implies a Role For Human V-Gamma/V-Delta-2 T Cells In Tumor Immunity. European Journal of Immunology 27, 3368-3379.

Girardi, M., Oppenheim, D. E., Steele, C. R., Lewis, J. M., Glusac, E., Filler, R., Hobby, P., Sutton, B., Tigelaar, R. E., and Hayday, A. C. (2001). Regulation of cutaneous malignancy by gammadelta T cells. Science 294, 605-9.

Gober, H. J., Kistowska, M., Angman, L., Jeno, P., Mori, L., and De Libero, G. (2003). Human T cell receptor gammadelta cells recognize endogenous mevalonate metabolites in tumor cells. J Exp Med 197, 163-8.

Groh, V., Steinle, A., Bauer, S., and Spies, T. (1998). Recognition of stress-induced MHC molecules by intestinal epithelial gammadelta T cells. Science 279, 1737-40.

Halary, F., Peyrat, M. A., Champagne, E., Lopezbotet, M., Moretta, A., Moretta, L., Vie, H., Fournie, J. J., and Bonneville, M. (1997). Control Of Self-Reactive Cytotoxic T Lymphocytes Expressing Gamma-Delta T Cell Receptors By Natural Killer Inhibitory Receptors. European Journal of Immunology 27, 2812-2821.

Harlow, E., and Lane, D. (1988). Antibodies: a laboratory manual (New York: Cold Spring Harbor Laboratory Ed.), pp. 312.

Lang, F., Peyrat, M. A., Constant, P., Davodeau, F., David-Ameline, J., Poquet, Y., Vie, H., Fournie, J. J., and Bonneville, M. (1995). Early activation of human V gamma 9V delta 2 T cell broad cytotoxicity and TNF production by nonpeptidic mycobacterial ligands. J Immunol 154, 5986-94.

Lutter, R., Saraste, M., van Walraven, H. S., Runswick, M. J., Finel, M., Deatherage, J. F., and Walker, J. E. (1993). F1F0-ATP synthase from bovine heart mitochondria: development of the purification of a monodisperse oligomycin-sensitive ATPase. Biochem J 295, 799-806.

Mahley, R. W., Hui, D. Y., Innerarity, T. L., and Beisiegel, U. (1989). Chylomicron remnant metabolism. Role of hepatic lipoprotein receptors in mediating uptake. Arteriosclerosis 9, I14-8.

Malkovska, V., Cigel, F., and Storer, B. E. (1994). Human T cells in hu-PBL-SCID mice proliferate in response to Daudi lymphoma and confer anti-tumour immunity. Clin. Exp. Immunol. 96, 158-165.

Malkovska, V., Cigel, F. K., Armstrong, N., Storer, B. E., and Hong, R. (1992). Antilymphoma activity of human gamma delta T-cells in mice with severe combined immune deficiency. Cancer. Res. 52, 5610-5616.

Martinez, L. O., Jacquet, S., Esteve, J. P., Rolland, C., Cabezon, E., Champagne, E., Pineau, T., Georgeaud, V., Walker, J. E., Terce, F., Collet, X., Perret, B., and Barbaras, R. (2003). Ectopic beta-chain of ATP synthase is an apolipoprotein A-I receptor in hepatic HDL endocytosis. Nature 421, 75-9.

Mezdour, H., Clavey, V., Kora, I., Koffigan, M., Barkia, A., and Fruchart, J. C. (1987). Anion-exchange fast protein liquid chromatographic characterization and purification of apolipoproteins A-I, A-II, C-I, C-II, C-III0, C-III1, C-III2 and E from human plasma. J Chromatogr 414, 35-45.

Mokuno, Y., Matsuguchi, T., Takano, M., Nishimura, H., Washizu, J., Ogawa, T., Takeuchi, O., Akira, S., Nimura, Y., and Yoshikai, Y. (2000). Expression of toll-like receptor 2 on gamma delta T cells bearing invariant V gamma 6/V delta 1 induced by Escherichia coli infection in mice. J Immunol 165, 931-40.

Moody, D. B., Reinhold, B. B., Guy, M. R,. Beckman, E. M., Frederique, D. E., Furlong, S. T., Ye, S., Reinhold, V. N., Sieling, P. A., Modlin, R. L., Besra, G. S., and Porcelli, S. A. (1997). Structural Requirements For Glycolipid Antigen Recognition By Cd1b-Restricted T Cells. Science 278, 283-286.

Morita, C. T., Beckman, E. M., Bukowski, J. F., Tanaka, Y., Band, H., Bloom, B. R., Golan, D. E., and Brenner, M. B. (1995). Direct presentation of nonpeptide prenyl pyrophosphate antigens to human gamma delta T cells. Immunity. 3, 495-507.

Moser, T. L., Stack, M. S., Asplin, I., Enghild, J. J., Hojrup, P., Everitt, L., Hubchak, S., Schnaper, H. W., and Pizzo, S. V. (1999). Angiostatin binds ATP synthase on the surface of human endothelial cells. Proc Natl Acad Sci USA 96, 2811-6.

Murao, K, Terpstra, V., Green, S. R., Kondratenko, N., Steinberg, D., and Quehenberger, O. (1997). Characterization of CLA-1, a human homologue of rodent scavenger receptor BI, as a receptor for high density lipoprotein and apoptotic thymocytes. J Biol Chem 272, 17551-7.

Sakaguchi, S. (2003). Control of immune responses by naturally arising CD4+ regulatory T cells that express toll-like receptors. J Exp Med 197, 397-401.

Spada, F. M., Grant, E. P., Peters, P. J., Sugita, M., Melian, A., Leslie, D. S., Lee, H. K., van Donselaar, E., Hanson, D. A., Krensky, A. M., Majdic, O., Porcelli, S. A., Morita, C. T., and Brenner, M. B. (2000). Self-recognition of CD1 by gamma/delta T cells: implications for innate immunity. J Exp Med 191, 937-48.

Tanaka, Y., Sano, S., Nieves, E., De Libero, G., Rosa, D., Modlin, R. L., Brenner, M. B., Bloom, B. R., and Morita, C. T. (1994). Nonpeptide ligands for human gamma delta T cells. Proceedings of the National Academy of Sciences of the USA 91, 8175-8179.

Wu, J., Groh, V., and Spies, T. (2002). T cell antigen receptor engagement and specificity in the recognition of stress-inducible MHC class I-related chains by human epithelial gamma delta T cells. J Immunol 169, 1236-40.

1-21. (canceled)

22. A method of assessing the sensitivity of a pathologic cell to γδT cell lysis or cytotoxicity or assessing whether a cell is a target cell for γδT cells, comprising determining in vitro or ex vivo whether said pathologic cell expresses, at the surface thereof, an ATP synthase polypeptide or a portion thereof, wherein the cell surface expression of an ATP synthase polypeptide or a portion thereof being an indication of the sensitivity of the pathologic cell to γδT cell lysis or cytotoxicity or wherein the cell surface expression of an ATP synthase polypeptide or a portion thereof being an indication that the cell is a target of γδT cells.

23. The method of claim 22, wherein said ATP synthase polypeptide is an ATP synthase α subunit.

24. The method of claim 22, wherein said ATP synthase polypeptide is an ATP synthase β subunit.

25. The method of claim 22, wherein said portion is an extracellular domain of an ATP synthase α or β subunit.

26. The method of claim 22, wherein said determination comprises incubating said cell, tissue or organ with a ligand specific for an ATP synthase polypeptide or a portion thereof and assessing binding of said ligand to said cell, tissue or organ.

27. The method of claim 26, wherein said ligand is an antibody or a fragment or derivative thereof.

28. The method of claim 22, wherein said pathologic cell is a tumor cell, a pathogen-infected cell, or a pathologic immune cell.

29. The method of claim 22, wherein said method assesses the sensitivity of a pathologic cell to γδT cell lysis or cytotoxicity.

30. The method of claim 22, wherein said method assesses whether a cell is a target cell for γδT cells.

31. A method of assessing the responsiveness of a patient to γδT cell-mediated therapy, or selecting patients for a γδT cell-mediated therapy program comprising determining whether said patient contains pathologic cells, tissues or organs that express, at the surface thereof, an ATP synthase polypeptide or a portion thereof, wherein the cell surface expression of an ATP synthase polypeptide or a portion thereof is an indication of the responsiveness of the patient to γδT cell-mediated therapy or wherein the cell surface expression of an ATP synthase polypeptide or a portion thereof is a criteria for selecting said patient for said γδT cell-mediated therapy program.

32. The method of claim 31, comprising providing a biopsy from said patient and determining cell surface expression of an ATP synthase polypeptide or a portion thereof by irnmunohistochemistry.

33. The method of claim 31, wherein said patient is a human subject.

34. The method of claim 31, wherein the γδT cell-mediated therapy comprises the injection of a drug that stimulates γδT cells in said patient.

35. The method of claim 31, wherein the γδT cell-mediated therapy comprises the injection of ex vivo activated γδT cells to said patient.

36. A method of stimulating γδT cells, comprising contacting γδT cells with:
a) an ATP synthase polypeptide or a TCR-binding fragment thereof;
b) an apolipoprotein A1 polypeptide or a variant or ATP synthase- or TCR-binding fragment thereof; or
c) an ATP synthase polypeptide or a TCR-binding fragment thereof linked to an apolipoprotein A1 polypeptide.

37. The method of claim 36, wherein said ATP synthase polypeptide or fragment or said ATP synthase polypeptide or a TCR-binding fragment thereof linked to an apolipoprotein A1 polypeptide is immobilized on a support.

38. The method of claim 36, wherein said γδT cells are human cells.

39. A method of increasing sensitivity of a target cell to γδT cell lysis or cytotoxicity, comprising causing or increasing cell surface expression of an ATP synthase polypeptide or a portion thereof in said target cell.

40. A method of screening, selecting or identifying a compound, comprising:
a) separately contacting γδT cells with an ATP synthase polypeptide or a TCR-binding fragment thereof in the presence and in the absence of a candidate compound; and
b) screening, selecting or identifying a compound that modulates activation of said γδT cells by said ATP synthase polypeptide or a TCR-binding fragment thereof.
